(19) United States
(12) Patent Application Publication
(10) Pub. No.: US 2023/0176076 A1
(43) Pub. Date: Jun. 8, 2023

(54) USE OF JULGI IN DIAGNOSING AND TREATING GUANINE-QUADRUPLEX-RELATED DISEASE

(71) Applicants: POSTECH RESEARCH AND BUSINESS DEVELOPMENT FOUNDATION, Pohang-si (KR); DAEGU GYEONGBUK INSTITUTE OF SCIENCE AND TECHNOLOGY, Daegu (KR)

(72) Inventors: Il Doo HWANG, Pohang-si (KR); Hyun Woo CHO, Cheongju-si (KR); Sung Bae LEE, Seoul (KR); Hyun Seob CHO, Gumi-si (KR)

(21) Appl. No.: 17/296,306

(22) PCT Filed: Nov. 27, 2019
(86) PCT No.: PCT/KR2019/016419

§ 371 Date: May 24, 2021

(30) Foreign Application Priority Data
Nov. 28, 2018 (KR) 10-2018-0149345

(51) Int. Cl. G01N 33/68; G01N 33/574; A61P 25/28; A61K 38/16; A61P 35/00

(52) U.S. Cl. CPC G01N 33/6896; A61K 38/168; A61P 25/28; A61P 35/00; G01N 33/574

(57) ABSTRACT

The present disclosure relates to a use of JULGI in diagnosing and treating guanine quadruplex-related disease and, more particularly, to a guanine-quadruplex (G-quadruplex) detecting composition comprising JULGI protein or a protein having a homology of 80% or higher to the JULGI protein, a diagnostic composition comprising the detecting composition, a diagnostic kit comprising the diagnostic composition, a method for providing information for diagnosis of a neurodegenerative disease, cancer, or a viral infection disease which are in pathological correlation with G-quadruplex, and a pharmaceutical composition for prevention or treatment of a neurodegenerative disease, cancer, or a viral infection disease, the pharmaceutical composition comprising JULGI protein, a protein having a homology of 80% or more with the JULGI protein, or a mutant protein thereof. Thanks to the activity of binding to the G-quadruplex structure, JULGI according to the present disclosure is expected to be advantageously used in diagnosing or treating various diseases that are in pathological correlation with the abnormal formation and dissociation of the G-quadruplex structure.

### TECHNICAL FIELD

The present disclosure describes to a use of JULGI in diagnosing and treating a guanine quadruplex-related disease, and more particularly, to a guanine-quadruplex (G-quadruplex) detecting composition including a JULGI protein or a protein having a homology of 80% or higher with the JULGI protein, a diagnostic composition including the detecting composition, a diagnostic kit including the diagnostic composition, a method for diagnosing a neurodegenerative disease, cancer, or a viral infection disease which has a pathological correlation with the G-quadruplex, and a use of the pharmaceutical composition, which includes a JULGI protein or a protein having a homology of 80% or more with the JULGI protein; or a mutant protein thereof, in preventing or treating a neurodegenerative disease, cancer, or a viral infection disease.

### BACKGROUND ART

Neurodegenerative diseases and cancer develop due to the accumulation of functional mutations in major genes caused by a wide range of influences, from genetic causes to environments. Up to now, various genes having a pathological correlation with the diseases have been identified, but the exact pathogenic mechanisms and causes of the diseases are yet to be clearly identified. In particular, there is no method for treating the neurodegenerative diseases, and the diseases are treated to an extent of delaying the progression of symptoms thereof. Therefore, research and development are required in various aspects spanning from molecular biological causes to diagnosis and treatment in order to basically treat the diseases.

Meanwhile, a guanine-quadruplex (G-quadruplex) structure is a secondary structure formed by Hoogsteen base pairing of four guanine bases. It has been reported that the abnormal formation or dissociation of the structure causes major diseases such as neurodegenerative diseases and cancer. In addition, it is known that the abnormal formation or dissociation of the structure has a close pathological correlation with various diseases. More particularly, it has been reported that an increase in a GGGGCC repeat base sequence present in the first intron of a C9orf72 gene is commonly observed in neurodegenerative diseases such as amyotrophic lateral sclerosis (ALS) and frontotemporal dementia (FTD), and a G-quadruplex structure formed from the transcript and the peptide formed by translation from the G-quadruplex structure are closely associated with the onset of the diseases. In addition to the C9orf72 gene, a G-quadruplex-forming repeat base sequence is also observed in genes and non-coding RNAs associated with various neurodegenerative diseases, indicating that the G-quadruplex structure has a close pathological correlation with neurodegenerative diseases (Roberto Simone et al., 2015, FEBS Letters).

Also, it is known that the proto-oncogenes closely associated with oncogenesis also have a G-quadruplex-forming base sequence at a major regulatory site that regulates the expression of genes, and this plays an important role in the regulation of gene expression. It is reported that a neuroblastoma RAS viral oncogene homolog (NRAS), which is a proto-oncogene, has a G-quadruplex-forming base sequence in the 5′UTR, and an increase in expression (translation) of the gene is caused by deletion or mutation of the G-quadruplex-forming base sequence. The presence of the G-quadruplex structure in the promoter regions on DNAs or the 5′UTR regions of RNAs of many proto-oncogenes means that gene expression regulation by regulating the formation of a G-quadruplex structure is important in cancer incidence. Also, the research results show that the functional mutagenesis of helicases and protein chaperones which regulate the formation of the G-quadruplex structure has an influence on cancer incidence, indicating that the regulation of formation of the G-quadruplex structure is closely associated with cancer.

### DISCLOSURE

### Technical Problem

Based on previous research results showing that the abnormal formation or dissociation of a guanine-quadruplex (G-quadruplex) structure is correlated with the onset of major diseases such as neurodegenerative diseases and cancer, the present inventors have experimentally confirmed that a plant-derived JULGI protein binding to a G-rich base sequence and a G-quadruplex structure may be used to diagnose or treat the related diseases. Therefore, the present disclosure has been completed based on this fact.

Therefore, the present disclosure describes a guanine-quadruplex (G-quadruplex) detecting composition which includes a JULGI protein or a protein having a homology of 80% or more with the JULGI protein.

Also, the present disclosure describes a composition for diagnosing a neurodegenerative disease, cancer, or a viral infection disease due to the guanine-quadruplex (G-quadruplex) binding activity of the JULGI protein, which includes the detecting composition, and a diagnostic kit including the diagnostic composition.

In addition, the present disclosure describes a method for providing information for diagnosis of a neurodegenerative disease, cancer, or a viral infection disease, which includes measuring the activity of a JULGI protein or a protein having a homology of 80% or more with the JULGI protein in a biological sample derived from a subject.

Additionally, the present disclosure describes a pharmaceutical composition for preventing or treating a neurodegenerative disease, which includes a JULGI mutant protein or gene encoding the JULGI mutant protein as an active ingredient.

Further, the present disclosure describes a pharmaceutical composition for preventing or treating cancer or a viral infection disease, which includes a JULGI protein or a protein having a homology of 80% or more with the JULGI protein; or a gene encoding the JULGI protein as an active ingredient.

However, the technical objects to be achieved by the present disclosure are not limited to the above-described technical objects, and other objects which are not mentioned above will be clearly understood from the following detailed description by those skilled in the art.

### Technical Solution

To achieve the objects of the present disclosure as described above, according to an aspect of The present disclosure, there is provided a guanine-quadruplex (G-quadruplex) detecting composition including a JULGI protein or a protein having a homology of 80% or more with the JULGI protein.

According to one embodiment of The present disclosure, the JULGI protein may have an amino acid sequence set forth in SEQ ID NO: 1 or 2.

According to another embodiment of The present disclosure, the JULGI protein may bind to a guanine-rich sequence or an RNA guanine-quadruplex.

According to another aspect of The present disclosure, there is provided a composition for diagnosing a neurodegenerative disease, cancer, or a viral infection disease due to the guanine-quadruplex (G-quadruplex) binding activity of the JULGI protein, which includes the detecting composition, and a diagnostic kit including the diagnostic composition.

According to still another aspect of The present disclosure, there is provided a method for providing information for diagnosis of a neurodegenerative disease, cancer, or a viral infection disease, which includes measuring the activity of a JULGI protein or a protein having a homology of 80% or more with the JULGI protein in a biological sample derived from a subject.

According to one embodiment of The present disclosure, the measuring of the activity may include determining whether the JULGI protein binds to a guanine-quadruplex

According to yet another aspect of The present disclosure, there is provided a pharmaceutical composition for preventing or treating a neurodegenerative disease, which includes a JULGI mutant protein or a gene encoding the JULGI mutant protein as an active ingredient.

According to one embodiment of The present disclosure, the JULGI mutant protein may have lost RNA binding activity because the arginine 20, 80, or 146 residue in an RanBP2-type zinc finger (ZnF) domain of the JULGI protein having an amino acid sequence set forth in SEQ ID NO: 1 is substituted with an alanine residue.

According to another embodiment of The present disclosure, the JULGI mutant protein may have lost RNA binding activity because the arginine 20, 79, or 152 residue in an RanBP2-type zinc finger (ZnF) domain of the JULGI protein having an amino acid sequence set forth in SEQ ID NO: 2 is substituted with an alanine residue.

According to still another embodiment of The present disclosure, the neurodegenerative disease may arise due to the guanine-quadruplex (G-quadruplex) structure.

According to yet another embodiment of The present disclosure, the neurodegenerative disease may include one or more selected from the group consisting of amyotrophic lateral sclerosis (ALS), frontotemporal dementia (FTD), progressive myoclonus epilepsy type 1 (EPM1), fragile X syndrome, spinocerebellar ataxia (SCA36), and familial Creutzfeldt-Jakob disease (CJD).

According to yet another aspect of The present disclosure, there is provided a pharmaceutical composition for preventing or treating cancer or a viral infection disease, which includes a JULGI protein or a protein having a homology of 80% or more with the JULGI protein; or a gene encoding the JULGI protein as an active ingredient.

According to one embodiment of The present disclosure, the cancer or the viral infection disease may arise due to the guanine-quadruplex (G-quadruplex) structure.

According to another embodiment of The present disclosure, the cancer may include one or more selected from the group consisting of pancreatic cancer, gastric cancer, liver cancer, colorectal cancer, colon cancer, rectal cancer, brain cancer, breast cancer, kidney cancer, thyroid cancer, bladder cancer, esophageal cancer, pharyngeal cancer, oral cavity cancer, tongue cancer, uterine cancer, ovarian cancer, leukemia, prostate cancer, testicular cancer, biliary tract cancer, gallbladder cancer, sarcoma, skin cancer, and lung cancer.

According to still another embodiment of The present disclosure, the viral infection disease may include one or more selected from the group consisting of acquired immunodeficiency syndrome caused by human immunodeficiency viruses, cervical cancer caused by human papilloma viruses, and Hodgkin’s lymphoma, Burkitt’s lymphoma, and nasopharyngeal carcinoma caused by the Epstein-Barr virus.

According to yet another aspect of The present disclosure, there is provided a method for preventing or treating a neurodegenerative disease, which includes administering a composition, which includes a JULGI mutant protein or a gene encoding the JULGI mutant protein as an active ingredient, to a subject.

According to yet another aspect of The present disclosure, there is provided a use of the JULGI mutant protein or the gene encoding the JULGI mutant protein for preventing or treating a neurodegenerative disease.

According to yet another aspect of The present disclosure, there is provided a method for preventing or treating cancer or a viral infection disease, which includes administering a composition, which comprises a JULGI protein or a protein having a homology of 80% or more with the JULGI protein; or a gene encoding the JULGI protein as an active ingredient, to a subject.

According to yet another aspect of The present disclosure, there is provided a use of the JULGI protein or the protein having a homology of 80% or more with the JULGI protein; or the gene encoding the JULGI protein for preventing or treating cancer or a viral infection disease.

### Advantageous Effects

According to The present disclosure, it was confirmed that a plant-derived JULGI protein having an activity of binding to a G-rich base sequence and a G-quadruplex structure binds to a gene including a G-quadruplex-forming base sequence, which has a pathological correlation with neurodegenerative diseases or cancer. In fact, an effect of the JULGI protein on symptoms of the diseases was confirmed in a Drosophila model of amyotrophic lateral sclerosis. Due to the activity of JULGI or a protein having a homology with the JULGI, JULGI is expected to be useful in diagnosing or treating various diseases which have a pathological correlation with the abnormal formation and dissociation of the G-quadruplex structure.

### DESCRIPTION OF DRAWINGS

FIG. 1A is a diagram showing a structure of a JULGI protein to identify a G-quadruplex motif that is a binding target of a JULGI protein.

FIG. 1B shows the results of performing SELEX analysis to determine whether the JULGI protein specifically binds to a G-rich base sequence and a G-quadruplex structure.

FIG. 2A shows the results of performing EMSA analysis using plant-derived SMXL5 having a G-rich base sequence that may form a G-quadruplex structure for JUL1 and its variant (i.e., JUL1(RA)) in order to confirm that the JULGI protein directly binds to an RNA G-quadruplex motif.

FIG. 2B shows the results of measuring a dissociation constant (Kd) of the binding of SMXL5 5’UTR to JUL1.

FIG. 2C shows the results of analyzing a surface interaction to visualize the interaction between an SMXL5 5’UTR G-quadruplex and a JUL1 protein.

FIG. 3A is a diagram showing G-quadruplex-forming sequences in a C9orf72 gene having a pathological correlation with a neurodegenerative disease, and four screened proto-oncogenes (for example, THRA, NRAS, FLI1, and BCL2).

FIG. 3B shows that a GST-JUL1 protein binds to a G-quadruplex-forming sequence in each of the genes.

FIG. 4 shows the results of analyzing an effect of the JULGI protein on the induction of symptoms of amyotrophic lateral sclerosis (ALS), that is, the results of comparing the degrees of amyotrophic lateral sclerosis after the JUL1 protein or a JUL1 mutant protein (JUL1^{R20/80/145A}) is expressed in a Drosophila model of amyotrophic lateral sclerosis.

FIG. 5 is a diagram showing that the JULGI protein is applicable to the diagnosis and treatment of neurodegenerative diseases, cancer, and viral infection diseases associated with an RNA G-quadruplex motif by using the characteristic that the JULGI protein directly binds to the RNA G-quadruplex motif.

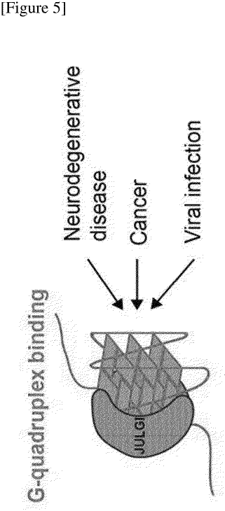

### BEST MODE

Based on previous research results showing that the abnormal formation or dissociation of an RNA guanine-quadruplex (G-quadruplex) structure is correlated with the onset of major diseases such as neurodegenerative diseases and cancer, the present inventors have confirmed that a plant-derived JULGI protein binding to a G-rich base sequence and a G-quadruplex structure may be used to diagnose or treat the related diseases. Therefore, the present disclosure has been completed based on this fact.

Therefore, the present disclosure provides an RNA guanine-quadruplex (G-quadruplex) detecting composition including a JULGI protein or a protein having a homology of 80% or more with the JULGI protein.

Also, the present disclosure provides a diagnostic composition including the detecting composition, characterized in that a neurodegenerative disease, cancer, or a viral infection disease is diagnosed due to the guanine-quadruplex (G-quadruplex) binding activity of the JULGI protein, and a diagnostic kit including the diagnostic composition.

In The present disclosure, the guanine-quadruplex (G-quadruplex) structure is a RNA secondary structure formed by Hoogsteen base pairing of four guanine bases. It has been reported that the abnormal formation or dissociation of the structure causes major diseases such as neurodegenerative diseases and cancer. In addition, it is known that the abnormal formation or dissociation of the structure has a close pathological correlation with various diseases.

In The present disclosure, the JULGI protein or the protein having a homology of 80% or more with the JULGI protein has an activity of binding to an RNA G-quadruplex structure. In this case, the protein may have an amino acid sequence set forth in SEQ ID NO: 1 or 2, and may more preferably include a JUL1 protein having an amino acid sequence set forth in SEQ ID NO: 1 or a JUL2 protein having an amino acid sequence set forth in SEQ ID NO: 2, and functional equivalents of the proteins. The term “functional equivalent” refers to a protein that has a sequence homology of at least 70% or more, preferably 80% or more, even more preferably 90% or more, and further preferably 95% or more with the amino acid sequence set forth in SEQ ID NO: 1 or 2 as a result of addition, substitution, or deletion of amino acids, that is, a protein that shows substantially the same physiological activity as the protein set forth in SEQ ID NO: 1 or 2. The term “substantially the same physiological activity” refers to an activity for forming a sieve tube in a plant.

The term “diagnosis” used in the present disclosure refers, in a broad sense, to the judgment of the actual condition of the patient’s disease in all aspects. The contents of the judgment include the name of a disease, the cause of a disease, the type and severity of a disease, detailed aspects of the conditions of a disease, the presence or absence of complications, and the like. In The present disclosure, the diagnosis is to judge the occurrence of a neurodegenerative disease, cancer, or a viral infection disease and a level of progression of the disease due to the guanine-quadruplex (G-quadruplex) binding activity of the JULGI protein.

A diagnostic kit of the present disclosure is composed of one or more different component compositions, solutions, or devices suitable for analytic methods.

The diagnostic kit of the present disclosure may include all types of various approaches using specific binding of JULGI to the G-quadruplex structure, and may be properly selected and prepared by those skilled in the art. For example, the cause of the corresponding disease and a degree of progression thereof may be diagnosed by applying the subject’s blood or tissue itself, an RNA sample extracted therefrom, or a DNA sample amplified by PCR to a diagnostic chip coated with the JULGI protein, coating the diagnostic chip with a tracer substance (such as Thioflavin T or NMM) capable of tracking a G-quadruplex structure present in the subject-derived sample, and quantifying a fluorescence signal. In this case, a bead surface interaction assay performed in Example 1-2 may be a basic concept of the diagnostic kit, but the present disclosure is not limited thereto.

The present inventors have confirmed that a JULGI protein may be used to diagnose or treat various diseases having a pathological correlation with the abnormal formation or dissociation of an RNA G-quadruplex structure according to specific embodiments.

According to one embodiment of The present disclosure, a G-quadruplex motif was confirmed as a binding target of the JULGI protein by performing SELEX analysis using various RNA probes for the JULGI protein (see Example 1-1). In addition, the realization of direct binding between an RanBP2-type ZnF motif of the JULGI protein and the G-quadruplex structure was confirmed by analysis using EMSA and a surface binding system using plant-derived SMXL5 having a G-rich base sequence capable of forming a G-quadruplex structure (see Example 1-2).

According to another embodiment of The present disclosure, a bead surface interaction assay was performed to verify whether JULGI binds to an RNA G-rich base sequence present in the 5’UTR of each of a C9orf72 gene having a pathological correlation with neurodegenerative diseases, and four screened proto-oncogenes (e.g., THRA, NRAS, FLI1, and BCL2). As a result, it is confirmed that the JULGI protein binds to each of the genes (see Example 2).

According to still another embodiment of The present disclosure, a JUL1 protein or a JUL1(RA) mutant protein having lost RNA binding activity of the JUL1 protein is expressed in a Drosophila model of amyotrophic lateral sclerosis in order to confirm an effect of JULGI on the induction of symptoms of amyotrophic lateral sclerosis. As a result, it is confirmed that the symptoms of amyotrophic lateral sclerosis significantly increased when the wild-type JUL1 protein is expressed, whereas the symptoms of amyotrophic lateral sclerosis significantly decreased compared to the control (G4C2)₃₀ when the JUL1(RA) is expressed (see Example 3).

The results according to the embodiments of the present disclosure demonstrate that JULGI may be used to specifically detect an increased level of the G-quadruplex structure for a neurodegenerative disease, cancer, a viral infection disease, and the like, which has a pathological correlation with the G-quadruplex structure, may be used to diagnose various diseases associated with the G-quadruplex structure, and may be useful in treating the related diseases using JULGI or variants thereof.

According to another aspect of the present disclosure, the present disclosure provides a method for providing information for diagnosis of a neurodegenerative disease, cancer, or a viral infection disease, which includes measuring the activity of a JULGI protein or a protein having a homology of 80% or more with the JULGI protein in a biological sample derived from a subject.

The term “method for providing information for diagnosis” used in the present disclosure refers to a preliminary stage for diagnosis which provides objective basic information necessary for the diagnosis of a neurodegenerative disease, cancer, or a viral infection disease or to predict the prognosis of the disease, excluding the physician’s clinical judgment or findings.

The neurodegenerative disease, the cancer, or the viral infection disease is characterized by having a pathological correlation with the G-quadruplex structure.

The neurodegenerative disease may preferably include one or more selected from the group consisting of amyotrophic lateral sclerosis (ALS), frontotemporal dementia (FTD), progressive myoclonus epilepsy type 1 (EPM1), fragile X syndrome, spinocerebellar ataxia (SCA36), and familial Creutzfeldt-Jakob disease (CJD), but the present disclosure is not limited thereto.

The cancer may preferably include one or more selected from the group consisting of pancreatic cancer, gastric cancer, liver cancer, colorectal cancer, colon cancer, rectal cancer, brain cancer, breast cancer, kidney cancer, thyroid cancer, bladder cancer, esophageal cancer, pharyngeal cancer, oral cavity cancer, tongue cancer, uterine cancer, ovarian cancer, leukemia, prostate cancer, testicular cancer, biliary tract cancer, gallbladder cancer, sarcoma, skin cancer, and lung cancer, but the present disclosure is not limited thereto.

The viral infection disease may preferably include one or more selected from the group consisting of acquired immunodeficiency syndrome caused by human immunodeficiency viruses, cervical cancer caused by human papilloma viruses, and Hodgkin’s lymphoma, Burkitt’s lymphoma, and nasopharyngeal carcinoma caused by the Epstein-Barr virus, but the present disclosure is not limited thereto.

In The present disclosure, the measuring of the activity of the JULGI protein or the protein having a homology of 80% or more with the JULGI protein may include determining whether the JULGI protein or a homologous protein thereof binds to a guanine-quadruplex structure. In this case, any method that is able to analyze an interaction between the protein and nucleic acids including RNA or DNA may be properly selected without any limitation to the type of method and performed by those skilled in the art.

In The present disclosure, the subject-derived biological sample may include a tissue, a cell, whole blood, blood, saliva, sputum, a cerebrospinal fluid, urine, and the like, but the present disclosure is not limited thereto.

According to still another aspect of the present disclosure, the present disclosure provides a pharmaceutical composition for preventing or treating a neurodegenerative disease, which includes a JULGI mutant protein or a gene encoding the JULGI mutant protein as an active ingredient.

The JULGI mutant protein may have lost RNA binding activity because the arginine 20, 80, or 146 residue in an RanBP2-type zinc finger (ZnF) domain of the JULGI protein (a JUL1 protein) having an amino acid sequence set forth in SEQ ID NO: 1; or the arginine 20, 79, or 152 residue in an RanBP2-type zinc finger (ZnF) domain of the JULGI protein (a JUL2 protein) having an amino acid sequence set forth in SEQ ID NO: 2 is substituted with another amino acid, preferably an alanine residue. Preferably, the JULGI mutant protein may have an amino acid sequence set forth in either SEQ ID NO: 5 or 6.

Also, the present disclosure provides a pharmaceutical composition for preventing or treating cancer or a viral infection disease, which includes a JULGI protein or a protein having a homology of 80% or more with the JULGI protein; or a gene encoding the JULGI protein as an active ingredient.

The neurodegenerative disease, the cancer, and the viral infection disease is characterized by having a pathological correlation with the guanine-quadruplex (G-quadruplex) structure.

The term “prevention” used in the present disclosure refers to all actions of inhibiting or delaying the onset of a neurodegenerative disease, cancer, or a viral infection disease by administration of the pharmaceutical composition according to the present disclosure.

The term “treatment” used in the present disclosure refers to all actions of improving or beneficially modifying the symptoms of the neurodegenerative disease, the cancer, or the viral infection disease by administration of the pharmaceutical composition according to the present disclosure.

In The present disclosure, the JULGI protein or the gene encoding the protein having a homology of 80% or more with the JULGI protein may include both genomic DNA and cDNA which encode the JULGI protein. Preferably, the gene of the present disclosure may be a JUL1 gene having a base sequence set forth in SEQ ID NO: 3, or a JUL2 gene having a base sequence set forth in SEQ ID NO: 4. The base sequence of SEQ ID NO: 3 is a sequence that encodes the protein having an amino acid sequence of SEQ ID NO: 1, and the base sequence of SEQ ID NO: 4 is a sequence that encodes the protein having an amino acid sequence of SEQ ID NO: 2. Also, variants of the base sequence fall within the scope of the present disclosure. Specifically, the gene may include a base sequence having a sequence homology of 70% or more, even more preferably 80% or more, further preferably 90% or more, and most preferably 95% or more to each of the base sequences set forth in SEQ ID NO: 3 and 4.

The “percentage (%) of sequence homology” with a polynucleotide is determined by comparing a comparison region with two optimally aligned sequences. In this case, some of a polynucleotide sequence in the comparison region may include an addition or a deletion (i.e., a gap), compared to a reference sequence (including no addition or deletion) for optimal alignment of the two sequences.

### Mode for Invention

Hereinafter, preferred embodiments of the present disclosure will be described only for a better understanding of the present disclosure. However, it should be understood that the following embodiments are provided to more easily understand the present disclosure, and are not intended to limit the scope of The present disclosure.

### EXAMPLES

### Example 1: Confirmation of Binding of JULGI Protein to G-Rich Base Sequence and G-Quadruplex Structure

### 1-1. Confirmation of G-Quadruplex Motif as Binding Target of JULGI

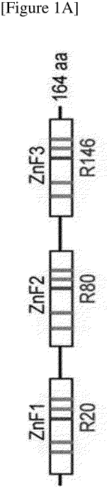

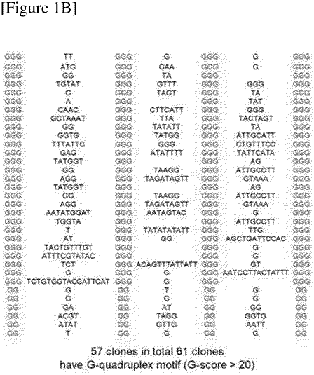

As shown in FIG. 1A, the JULGI protein has three RanBP2-type ZnF domains, and each of the domains has a conserved arginine residue (R20, R80, and R146 present in ZnF1, ZnF2, and ZnF3, respectively) necessary for RNA binding. Therefore, the present inventors performed SELEX analysis to determine whether the JULGI protein having a structure as described above specifically binds to a G-rich base sequence and a guanine-quadruplex (G-quadruplex) structure. An RNA probe bound to the JULGI protein was optionally concentrated, and a base sequence was confirmed. As a result, as shown in FIG. 1B, it was confirmed that the RNA bound to the JULGI included a guanine (G)-rich sequence, and mainly formed G-quadruplexes (having a high G-score (a G-score of greater than 20) in 57 of the 61 RNAs), and the secondary structure formed Hoogsteen-bonded G-quartet stacks.

### 1-2. Confirmation of G-Quadruplex Formation Through Direct Binding Between JULGI Protein and RNA G-Quadruplex Motif

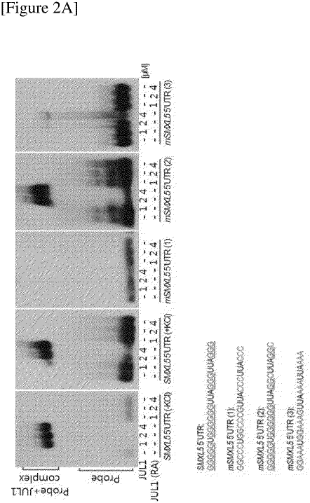

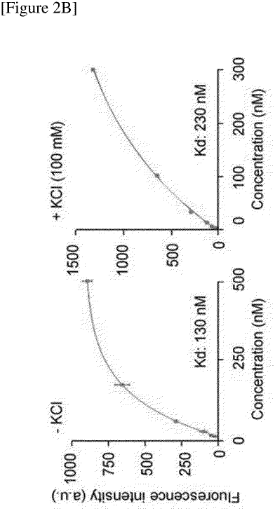

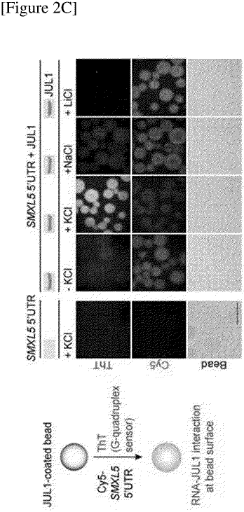

Based on the results, EMSA was performed using GST-JUL1 and GST-JUL2 proteins under conditions with or without potassium necessary for G-quadruplex formation in order to determine whether the JULGI protein directly binds to a single-stranded mRNA or RNA G-quadruplex structure using plant-derived SMXL5 having a G-rich base sequence capable of forming a G-quadruplex structure. As a result, as shown in FIG. 2A, it was observed that JUL1 delayed the migration of the G-quadruplex-forming motif by binding to an SMXL5 5’UTR probe having a G-quadruplex-forming motif used as the positive control. However, the above-described results were not observed in the case of mSMXL5 5’UTR (1), mSMXL5 5’UTR (3) having no G-quadruplex-forming motif. On the other hand, it was confirmed that mSMXL5 5’UTR (2), which is a variant forming two G-quartet layers, was bound to the JUL1 protein although it was less effectively bound to the JUL1 protein, compared to the SMXL5 5’UTR. Also, a dissociation constant (Kd) of the binding of JUL1 to SMXL5 5’UTR was measured. As a result, as shown in FIG. 2B, the dissociation constant (Kd) was estimated to be 130 nM and 230 nM, respectively, in the presence and absence of potassium, indicating that JUL1 preferentially binds to the primary SMXL5 5’UTR sequence.

Next, to visualize the interaction between the SMXL5 5’UTR G-quadruplex and the JUL1 protein, an experiment was performed using a surface binding system condensed with potassium and sodium which strongly and weakly stabilize the G-quadruplex for an RNA probe at the liquid-solid interface of the protein-coated bead surface, respectively, and with lithium which does not stabilize the G-quadruplex. For this purpose, specifically, as shown in FIG. 2C, glutathione-sepharose beads coated with GST-JUL1 was treated with a Cy5-labeled RNA probe (i.e., SMXL5 5’UTR). Accordingly, the G-quadruplex structure formed on the surface of the bead was visualized at the same time using the Cy5-labeled RNA probe and the G-quadruplex sensor (i.e., Thioflavin T (ThT)). As a result, as shown in FIG. 2C, a fluorescence signal emitted by ThT appeared to completely overlap with that of the JUL1-coated bead surface in the presence of potassium (+KCl), but the fluorescence signal decreased in the presence of sodium (+NaCl) or lithium (+LiCl). On the other hand, the Cy5 fluorescence overlapping with that of the bead surface increased in the presence of sodium or lithium. These results indicate that the direct binding of JUL1 to RanBP2-type ZnF occurs in the G-quadruplex structure of SMXL5 5’UTR.

### Example 2: Confirmation of Binding of JULGI Protein to G-Rich Base Sequence and G-Quadruplex Structure in Gene Associated With Onset of Neurodegenerative Disease and Cancer

Based on the results of Example 1, the present inventors endeavored to determine whether the JULGI protein binds to the RNA G-rich base sequence and the G-quadruplex structure in genes associated with the onset of a disease such as a neurodegenerative disease or cancer. For this purpose, it was verified by a bead surface interaction assay whether the JULGI protein binds to the RNA G-rich base sequence present in the 5’UTR of each of a C9orf72 gene having a pathological correlation with a neurodegenerative disease, and four screened proto-oncogenes (for example, THRA, NRAS, FLI1, and BCL2).

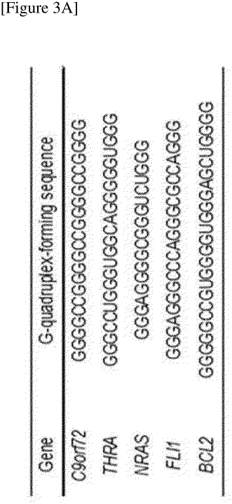

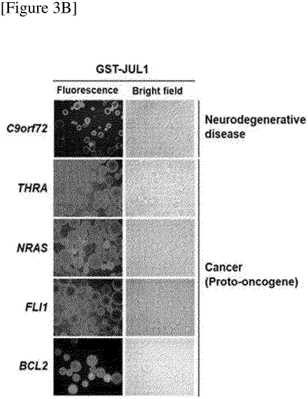

As shown in FIG. 3A, it can be seen that each of the five genes had the G-quadruplex-forming sequence. Based on the experimental results, it was confirmed by observation under a fluorescence microscope that the GST-fused JUL1 protein (GST-JUL1) binds to each of the genes, as shown in FIG. 3B. The results showed that the JULGI protein specifically detects the G-quadruplex structure increased due to the onset of a neurodegenerative disease, cancer, or the like, and can be effectively used for diagnosing various diseases associated with the G-quadruplex structure.

### Example 3: Confirmation of Effect of JULGI Protein on Induction of Symptoms of Amyotrophic Lateral Sclerosis

In addition to the results of Example 2, the present inventors endeavored to determine an effect of the JULGI protein on the induction of the symptoms of amyotrophic lateral sclerosis (ALS) which is one neurodegenerative disease. For this purpose, the JUL1 protein, and JUL1(RA) whose RNA binding activity was eliminated by replacing an arginine residue present in an Ranbp2 type zinc finger domain of the JUL1 protein with an alanine residue were expressed in a Drosophila model of amyotrophic lateral sclerosis.

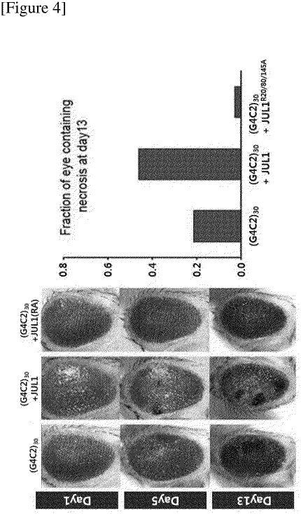

As a result, as shown in FIG. 4, it was confirmed that the symptoms of amyotrophic lateral sclerosis significantly increased when the wild-type JUL1 protein was expressed, whereas the symptoms of amyotrophic lateral sclerosis significantly decreased when JUL1^{R20/80/145A} was expressed, compared to the control (G4C2)₃₀. These results show that the symptoms of amyotrophic lateral sclerosis are able to be controlled using JULGI, which is a G-quadruplex binding protein and a G-quadruplex folder, and that various diseases having a pathological correlation with the G-quadruplex (including amyotrophic lateral sclerosis) are able to be treated using JULGI(RA).

The foregoing description of the present disclosure is intended for illustration, and thus it will be understood by those skilled in the art to which the present disclosure pertains that the present disclosure may be easily modified in other specific forms without changing the technical scope or essential features of The present disclosure. Therefore, it should be understood that the embodiments described above are illustrative in all respects and are not intended to limit The present disclosure.

### Industrial Applicability

The JULGI protein or the protein having homology with the JULGI protein is expected to be advantageously used as a diagnostic kit and a therapeutic agent to treat various diseases (for example, neurodegenerative diseases, cancer, or viral infection diseases) having a pathological correlation with the abnormal formation and dissociation of a G-quadruplex structure as known in the related art.

1. A guanine-quadruplex (G-quadruplex) detecting composition comprising a JULGI protein or a protein having a homology of 80% or more with the JULGI protein.

2. The detecting composition of claim 1, wherein the JULGI protein has an amino acid sequence set forth in SEQ ID NO: 1 or 2.

3. The detecting composition of claim 1, wherein the JULGI protein binds to a guanine-rich sequence or an RNA guanine-quadruplex.

4. A diagnostic composition comprising the composition defined in claim 1,
wherein a neurodegenerative disease, cancer, or a viral infection disease is diagnosed due to the guanine-quadruplex (G-quadruplex) binding activity of the JULGI protein.

5. A diagnostic kit comprising the composition defined in claim 4.

6. A method for providing information for diagnosis of a neurodegenerative disease, cancer, or a viral infection disease, the method comprising:
measuring the activity of a JULGI protein or a protein having a homology of 80% or more with the JULGI protein in a biological sample derived from a subject.

7. The method of claim 6, wherein the JULGI protein has an amino acid sequence set forth in SEQ ID NO: 1 or 2.

8. The method of claim 6, wherein the measuring of the activity comprises determining whether the JULGI protein binds to a guanine-quadruplex structure.

9. A method for preventing or treating a neurodegenerative disease, comprising:
administering a pharmaceutical composition, which comprises a JULGI mutant protein or a gene encoding the JULGI mutant protein as an active ingredient, to a subject.

10. The method of claim 9, wherein the JULGI mutant protein has lost RNA binding activity because the arginine 20, 80, or 146 residue in an RanBP2-type zinc finger (ZnF) domain of the JULGI protein having an amino acid sequence set forth in SEQ ID NO: 1 is substituted with an alanine residue.

11. The method of claim 9, wherein the JULGI mutant protein has lost RNA binding activity because the arginine 20, 79, or 152 residue in an RanBP2-type zinc finger (ZnF) domain of the JULGI protein having an amino acid sequence set forth in SEQ ID NO: 2 is substituted with an alanine residue.

12. The method of claim 9, wherein the neurodegenerative disease arises due to a guanine-quadruplex (G-quadruplex) structure.

13. The method of claim 9, wherein the neurodegenerative disease comprises one or more selected from the group consisting of amyotrophic lateral sclerosis (ALS), frontotemporal dementia (FTD), progressive myoclonus epilepsy type 1 (EPM1), fragile X syndrome, spinocerebellar ataxia (SCA36), and familial Creutzfeldt-Jakob disease (CJD).

14. A method for preventing or treating cancer or a viral infection disease, the method comprising:
administering a pharmaceutical composition, which comprises a JULGI protein or a protein having a homology of 80% or more with the JULGI protein; or a gene encoding the JULGI protein as an active ingredient, to a subject.

15. The method of claim 14, wherein the JULGI protein has an amino acid sequence set forth in SEQ ID NO: 1 or 2.

16. The method of claim 14, wherein the cancer or the viral infection disease arises due to a guanine-quadruplex (G-quadruplex) structure.

17. The method of claim 14, wherein the cancer comprises one or more selected from the group consisting of pancreatic cancer, gastric cancer, liver cancer, colorectal cancer, colon cancer, rectal cancer, brain cancer, breast cancer, kidney cancer, thyroid cancer, bladder cancer, esophageal cancer, pharyngeal cancer, oral cavity cancer, tongue cancer, uterine cancer, ovarian cancer, leukemia, prostate cancer, testicular cancer, biliary tract cancer, gallbladder cancer, sarcoma, skin cancer, and lung cancer.

18. The method of claim 14, wherein the viral infection disease comprises one or more selected from the group consisting of acquired immunodeficiency syndrome caused by human immunodeficiency viruses, cervical cancer caused by human papilloma viruses, and Hodgkin’s lymphoma, Burkitt’s lymphoma, and nasopharyngeal carcinoma caused by the Epstein-Barr virus.

19-22. (canceled)


SEQUENCE LISTING (11 sequences)

SEQ ID NO: 1 - protein, 164 aa, Arabidopsis thaliana
MSRPGDWNCRSCSHLNFQRRDSCQRCGDSRSGPGGVGGLDFGNFGGRAMSAFGFTTGSDVRPGDWYCTVGNCGTHNFASRSTCFKCGTFKDETGAGGGGGGIGGPAMFDADIMRSRVPGNGGRSSWKSGDWICTRIGCNEHNFASRMECFRCNAPRDFSNRTSF

SEQ ID NO: 2 - protein, 170 aa, Arabidopsis thaliana
MNRPGDWNCRLCSHLNFQRRDSCQRCREPRPGGISTDLLSGFGGRPVSSSFGFNTGPDVRPGDWYCNLGDCGTHNFANRSSCFKCGAAKDEFSCSSAAATTGFMDMNVGPRRGLFGFGGSSSGGGGTGRSPWKSGDWICPRSGCNEHNFASRSECFRCNAPKELATEPPY

SEQ ID NO: 3 - DNA, 495 bp, Arabidopsis thaliana
atgagcagacccggagattggaactgcaggtcatgcagccatctcaacttccagcgccgtgactcttgccagcgatgcggtgactctcgttccggccccggtggagttggtggcttagactttggtaatttcggtggcagagccatgtctgctttcggattcaccaccggctccgacgttcgtcccggtgattggtactgcaccgtgggaaactgcgggacacacaacttcgccagtcgctccacctgcttcaaatgcggcactttcaaggacgagaccggcgctggaggcggaggtggtggcatcggcggtccggccatgtttgacgccgacattatgcggtctagagtccccggtaacggtggtcgctctagctggaaatccggcgactggatttgcactaggattggttgcaatgagcataactttgcaagcagaatggaatgcttcaggtgcaatgcaccaagggacttcagcaacagaacctctttctaa

SEQ ID NO: 4 - DNA, 513 bp, Arabidopsis thaliana
atgaataggccgggagattggaactgcagattgtgtagccacctcaacttccagaggagggattcatgccaacgttgtagagagcctagaccgggcgggatcagtaccgatttactcagcggttttggtggccgtccggttagtagctccttcggtttcaacaccgggcccgatgtgcgacccggggattggtattgcaaccttggggattgtgggacacataattttgccaataggtccagttgtttcaagtgtggtgccgcaaaagatgagttttcatgctcaagtgctgctgcaacaaccgggtttatggacatgaatgttggtccgagacgtggcctttttggttttggcggcagcagtagtggtggtggtggtacgggccgttctccttggaaatctggagattggatttgcccaaggtcaggctgtaacgaacataacttcgcaagcaggtcagagtgtttcaggtgtaacgcaccaaaggaacttgccaccgaaccaccctattag

SEQ ID NO: 5 - protein, 164 aa, Arabidopsis thaliana
MSRPGDWNCRSCSHLNFQRADSCQRCGDSRSGPGGVGGLDFGNFGGRAMSAFGFTTGSDVRPGDWYCTVGNCGTHNFASASTCFKCGTFKDETGAGGGGGGIGGPAMFDADIMRSRVPGNGGRSSWKSGDWICTRIGCNEHNFASAMECFRCNAPRDFSNRTSF

SEQ ID NO: 6 - protein, 170 aa, Arabidopsis thaliana
MNRPGDWNCRLCSHLNFQRADSCQRCREPRPGGISTDLLSGFGGRPVSSSFGFNTGPDVRPGDWYCNLGDCGTHNFANASSCFKCGAAKDEFSCSSAAATTGFMDMNVGPRRGLFGFGGSSSGGGGTGRSPWKSGDWICPRSGCNEHNFASASECFRCNAPKELATEPPY

SEQ ID NO: 7 - DNA, 22 bp, Artificial Sequence
  G-quadruplex-forming sequence_C9orf72
ggggccggggccggggccgggg

SEQ ID NO: 8 - DNA, 22 bp, Artificial Sequence
  G-quadruplex-forming sequence_THRA
gggccuggguggcagggguggg

SEQ ID NO: 9 - DNA, 18 bp, Artificial Sequence
  G-quadruplex-forming sequence_NRAS
gggaggggcgggucuggg

SEQ ID NO: 10 - DNA, 22 bp, Artificial Sequence
  G-quadruplex-forming sequence_FLI1
gggagggcccagggcgccaggg

SEQ ID NO: 11 - DNA, 25 bp, Artificial Sequence
  G-quadruplex-forming sequence_BCL2
gggggccguggggugggagcugggg